(19) United States
(12) Patent Application Publication
(10) Pub. No.: US 2023/0270696 A1
(43) Pub. Date: Aug. 31, 2023

(54) MEDICINAL USE OF FINGOLIMOD IN PREVENTION AND TREATMENT OF NEURODEGENERATIVE DISEASES CAUSED BY SPHINGOLIPID DISORDERS

(71) Applicant: Dan CHEN, Hangzhou (CN)

(72) Inventor: JUN-PING LIU, Hangzhou (CN)

(21) Appl. No.: 18/314,781

(22) Filed: May 9, 2023

Related U.S. Application Data
(63) Continuation of application No. PCT/CN2021/118796, filed on Sep. 16, 2021.

(30) Foreign Application Priority Data
Nov. 9, 2020 (CN) 202011239044.1

(51) Int. Cl. A61K 31/137; A61P 25/28; A61K 9/00

(52) U.S. Cl. CPC A61K 31/137; A61P 25/28; A61K 9/0053

(57) ABSTRACT

Provided is a medicinal use of fingolimod as sphingomyelinase inhibitor in preventing and treating neurodegenerative diseases caused by sphingolipid disorders. Research has shown that fingolimod can effectively alleviate and significantly improve the motion function of spastic paraplegic mice, enable old paraplegic mice to stand again, and increase the number of times paraplegic mice stand. Meanwhile, the present invention can effectively reduce/lower lipofuscin deposition and axonal myelin sheath tear in mouse brain tissue, can significantly alleviate the reduction or deficiency of sphingomyelin, promote the recycling of sphingomyelin, and correct neurological dysfunction caused by sphingolipid disorders that are due to a sphingomyelin deficiency. Further provided is an application of fingolimod in the preparation of a drug for preventing and treating neurodegenerative diseases caused by sphingolipid disorders.

### CROSS REFERENCE

The present disclosure is a continuation-application of International (PCT) Patent Application No. PCT/CN2021/118796, filed on Sep. 16, 2021, which claims priority of Chinese Patent Application No. 202011239044.1, filed on Nov. 9, 2020, the entire contents of which are hereby incorporated by reference in their entirety.

### TECHNICAL FIELD

The present disclosure belongs to the field of pharmaceutical biotechnology and relates to the medicinal use of fingolimod (FTY720) as a novel sphingomyelinase inhibitor in the prevention and treatment of neurodegenerative diseases caused by sphingolipid disorders, which refers to pharmaceutical use of FTY720 (fingolimod) in the preparation of the treatment of neurodegenerative diseases caused by sphingolipid disorders such as early-onset Parkinson's syndrome and dementia (Kufor-Rakeb syndrome or KRS), hereditary spastic paraplegia (HSP, spastic paraplegia or SPG), amyotrophic lateral sclerosis (ALS, motor neuron disease or MND), corticobasal degeneration (CBD), spinal cerebellar ataxia (SCA), and neuronal ceroid lipofuscinosis (NCL).

### BACKGROUND

FTY720 (fingolimod) has the molecular formula C₁₉H₃₃NO₂·HCl and the chemical name 2-amino-2-[2-(4-octylphenyl)]-1,3-propanediol hydrochloride with a molecular weight of 343.94 g/mol. Phosphorylated fingolimod exerts its effect by binding to S113 receptors [Hla T, et al. Science, 2001], thereby confining lymphocytes to lymph nodes and preventing them from entering the central nervous system, thus providing central nervous system protection. Fingolimod has also been used to suppress post-transplant immune rejection because of its immunosuppressive effects [Park S I, et al. Braz J Med Biol Res, 2005] and has been approved by the US Food and Drug Administration (FDA) as the first oral drug for the treatment of relapsing multiple sclerosis (MS). Thus, fingolimod is currently known to improve neurological deficits by modulating inflammatory and immune processes, and its therapeutic effects do not act directly on nerve cells.

Recently, gene-disease association studies have shown that mutations in the ATP13A2 gene are strongly associated with the development of Parkinson's disease, especially early-onset Parkinson's syndrome and dementia (Kufor-Rakeb syndrome (KRS); MIM606693) [Fleming, S. M., et al. 2018]. In addition, the ATP13A2 mutations are associated with hereditary spastic paraplegia (HSP/SPG) [Estrada-Cuzcano, A., Brain, 2017], with neuronal ceroid lipofuscinoses (NCL) [Schultheis, P. J., et al. Human molecular genetics, 2013], and also with amyotrophic lateral sclerosis (ALS) [Spataro, R., et al. Hum Genomics, 2019]. However, the causal relationship between the ATP13A2 mutations and these above movement disorders, and how to target interventions in the prevention and treatment strategies require further investigation.

Spastic paraplegia (SPG) is a group of neurodegenerative disorders that often present with an extrapyramidal syndrome-like appearance with progressive limb paralysis and lower limb spasticity. In addition to limb spasticity and weakness, symptoms include epilepsy, deafness, cerebellar dysfunction, cognitive impairment, visual impairment, and peripheral neuropathy. There is increased muscle tone in both lower extremities, active hyperactive tendon reflexes, positive pathological reflexes, and a scissor gait. More than 70 different types are known, including all modes of inheritance of autosomal dominant, autosomal recessive, X-linked or non-Mendelian mitochondrial matrilineal inheritance. Mutations in the ATP13A2 gene are an important causative gene for SPG. Progressive exacerbation of SPG severely affects patients' ability to work and care for themselves, and there is no effective therapy to prevent, terminate, or reverse the disease, except through pharmacological, physical, or surgical treatment to relieve patients' symptoms. The pathological changes are mainly in the axonal degeneration, demyelination and/or impaired remyelination of the bilateral corticospinal tracts in the spinal cord, often most severely in the thoracic segment.

Amyotrophic lateral sclerosis (ALS) remains an incurable and fatal neurodegenerative disease. Recently, mutations in ATP13A2 have been identified as an important pathogenic injury in ALS, causing damage to both upper and lower motor neurons, which results in muscle paralysis, including progressive muscle weakness and atrophy in the bulb (part of the muscle innervated by the medulla oblongata), extremities, trunk, chest and abdomen. The cause of ALS is still unclear, and the disease progresses slowly in some patients but more rapidly in others, from weakness of the limb muscles to weakness of the thoracic respiratory muscles within a few months, and then to respiratory failure, threatening the life of the patient. There is no specific therapy for this disease, and the therapeutic effect of riluzole for this disease varies from person to person and is not ideal, with some patients showing ineffectiveness of oral riluzole therapy.

Corticobasaldegeneration (CBD) is a chronic progressive neurodegenerative disease characterized by asymmetric episodes of akinetic tonic syndrome, disuse, dystonia, and postural abnormalities. Progressive Parkinson's syndrome with markedly asymmetric signs and symptoms of cortical and basal ganglia damage is seen clinically, and its pathological alteration involves the accumulation of abnormal tau proteins in neurons and glial cells.

Spinal cerebellar ataxia is a genetic disorder with movement disorders as the main symptom. Pathologically, the manifestations are diverse, with common atrophy and degeneration of nerve cells, loss of axonal myelin sheaths, and mild proliferation of glial cells. Extensive degeneration of the cerebellar hemispheres, cerebellar vermis, the middle and lower cerebellar peduncles occurred with losses of Purkinje cells. There are atrophy or loss of nerve cells in the posterior columns of the spinal cord and Clark's column, secondary to Glial cell hyperplasia, degeneration and axonal myelin sheath loss in the posterior roots and spinal ganglia, especially in the lumbar and sacral segments of the spinal cord. There is no cure to these disorders and the disease remains incurable.

Neuronal ceroid lipofuscinoses (NCL) is a group of at least 13 different, progressive neurodegenerative diseases that are associated with CLN genes 1-14 (CLN9 is also a CLN5 mutation). Mutations in ATP13A2 are the most recently identified pathogenic gene. NCL features include brain degeneration and deposition of lysosomal autofluorescent storage material (called lipofuscin). NCLs are commonly autosomal recessive disorders with childhood onset that manifest as muscle twitching episodes, visual impairment, mental decline, and early death. Adult-onset NCL is not always CLN4 type (autosomal dominant Kufs disease or Parry disease), and recessive-onset CLN6 (MIM 601780) and CLN5 mutations also occur. Most adult-onset patients are between the ages of 20 and 40 years and present with muscle twitching spastic seizures. Mild features include behavioral changes, progressive tremor, myoclonus, memory loss, and frequent falls. Seizures are followed by a slow, progressive cognitive decline, ataxia, memory loss and speech disturbances. Some patients show signs of depression. The age of death is usually between the late 30s and 60 years, and the cause of death is severe neurological damage and multiorgan failure.

Sphingomyelin is a major component of the nerve myelin sheath and cell membrane structure, and is the main phospholipid for the synthesis of sphingolipids such as cerebrosides and gangliosides, and for the production of other sphingolipids such as ceramide and sphingosine. Currently, it is known that sphingolipids are synthesized by the endoplasmic reticulum and transported via the Golgi apparatus to the cell membrane, where they are internalized and degraded by lysosomes. However, we show that sphingomyelin deficiency due to lysosomal sphingomyelin storage and sphingomyelinase-mediated degradation causes neurodegenerative diseases.

### SUMMARY OF THE DISCLOSURE

The purpose of the present disclosure is to provide the medicinal use of fingolimod (FTY720) in the prevention and treatment of neurodegenerative diseases caused by sphingolipid disorders, that is, to provide the application of fingolimod (FTY720) in the preparation of drugs for the prevention and treatment of neurodegenerative diseases caused by sphingolipid disorders, specifically sphingomyelin lysosomal storage with ceramide spillover disorders. It was found that FTY720 is a sphingomyelinase inhibitor, regulating sphingolipid metabolism, i.e., FTY720 can directly inhibit sphingomyelinase activity and regulate the sphingolipid metabolism of nerve cells, thus protecting against nerve cell loss of function caused by disorders of sphingolipid metabolism. Further study found that FTY720 can effectively reduce lipofuscin deposition and axonal myelin sheath tearing in brain tissue, i.e., it has a direct protective effect on nerve cells by regulating nerve cell sphingolipid metabolism. While KRS, SPG, ALS, corticobasal degeneration, spinal cerebellar ataxia and neuronal ceroid lipofuscinosis have neurodegenerative lesions related to ATP13A2 mutations and sphingomyelin deficiency with disorders of sphingolipid metabolism, the FTY720 drug provided by the present disclosure has an alleviative beneficial effect on the above neurodegenerative diseases through the regulation of sphingolipid metabolism.

The neurodegenerative diseases include KRS, SPG, ALS, corticobasal degeneration, spinal cerebellar ataxia, and neuronal ceroid lipofuscinosis, as well as motor neuron diseases with disorders of sphingolipid metabolism due to reduced or deficient sphingomyelin, or impaired sphingomyelin recycling from lysosome to cell membrane.

A route of administration of the drugs includes oral administration, intraperitoneal administration, and ventricular injection administration.

The drugs are made from an effective dose of FTY720 and pharmaceutically permissible excipients. A total mass fraction of FTY720 in the drugs is 0.01-10%.

During preparation of the drugs, drug powder of FTY720 is dissolved in 0.9% normal saline and administered in a concentration range of 0.01-5 mg/kg.

To solve the problems in the related art, the present disclosure provides a novel drug function as sphingomyelinase inhibitor for the prevention and treatment of neurodegenerative diseases such as KRS, SPG, ALS, corticobasal degeneration, spinal cerebellar ataxia, and neuronal ceroid lipofuscinosis, which was originally used as a clinical first-line immunosuppressive drug with a few reports of anticancer treatment and has not yet been used for aforementioned neurodegenerative movement disorders such as KRS, SPG, ALS and NCL. Experimental studies of the present disclosure through different routes and different doses of therapeutic administration show that FTY720 can effectively alleviate and significantly improve the motor function of spastic paraplegic mice, and FTY720 can make aged paralytic mice stand up again and increase the number of standing in paralytic mice. In addition, FTY720 can effectively reduce lipofuscin deposition and axonal myelin sheath tearing in mouse brain tissue, FTY720 can significantly alleviate sphingomyelin reduction or deficiency, promote the sphingolipid recycling, and correct neurological dysfunction caused by the sphingolipid deficiency. Investigations show that FTY720 is a sphingomyelinase inhibitor, thereby inhibiting sphingomyelin degradation or breakdown to ceramide.

### BRIEF DESCRIPTION OF THE DRAWINGS

FIG. 1: The left figure shows a comparison of the content of each lipid component in wild-type control (Ctrl) and pathological model (ATP13A2 knockdown or KD) SH-SY5Y nerve cells. The horizontal coordinate is the value of log 2 (KD/Ctrl), indicating the fold difference. The vertical coordinate is −log 10 (statistical p-value), according to which the graph visualizes the lipids with significant differences between the two groups. This shows that the sphingomyelin (SM) of the different carbon chains is significantly increased in the pathological model group in both groups of cells transiently deficient in ATP13A2, indicating SM lysosomal storage. The right figure of FIG. 1 shows the heat map of the different SM contents in wild-type (Ctrl) and ATP13A2 KD pathological model (KD) SH-SY5Y nerve cells. It can also be seen that the SM content is significantly increased in the ATP13A2 KD group compared to Ctrl.

FIG. 2 shows the content of sphingomyelin (SM-C16 and SM-C18), ceramide (Cer-C16 and Cer-C18), and sphingosine (Sph) in lysosomes in wild-type (Ctrl) and pathological model (ATP13A2 KD) SH-SY5Y nerve cells. This shows that lysosomal sphingomyelin is significantly increased in the pathological model cells deficient in ATP13A2.

FIG. 3 shows the content of each lipid in the nigrostriatal site of wild-type and ATP13A2 gene knockout (KO) pathological model mice. This shows that the sphingomyelin (SM) content is significantly reduced and the sphingomyelin breakdown product—ceramide (Cer) is significantly increased in the pathological model group.

FIG. 4 shows the content of each lipid in the orbitofrontal cortex site of wild-type and ATP13A2 KO pathological model mice. This shows that the sphingomyelin (SM) content is significantly reduced and the sphingomyelin breakdown product—ceramide (Cer) is significantly increased in the pathological model group.

FIG. 5 shows the content of each lipid in the hypothalamus site of wild-type and ATP13A2 KO pathological model mice. This shows that the sphingomyelin (SM) content is significantly reduced and the sphingomyelin breakdown product—ceramide (Cer) is significantly increased in the pathological model group.

FIG. 6 shows a comparison of the content of each lipid component in wild-type (Ctrl) and ATP13A2 KO pathological model (KO) MEF cells. The horizontal coordinate is the value of log 2 (ATP13A2 KO/Ctrl) and the vertical coordinate is −log 10 (statistical p-value). The graph visualizes the lipid classes with significant differences between the two groups. This shows that the sphingomyelin (SM) content and the contents of gangliosides (GM1, GM2 and GM3) are significantly reduced, whereas ceramide (Cer) and cerebrosides (GluCer and LacCer) are significantly reduced in the pathological model group cells.

FIG. 7 shows the values of absorbance proportional to the enzyme activity of sphingomyelinase (vertical axis) with sphingomyelin as substrate. Different concentrations (horizontal axis) of purified sphingomyelinase are added in the presence or absence of FTY720 and incubation proceeded for 60 min at 37° C., before measuring sphingomyelin breakdown in absorbance. Blue line (top line): value of sphingomyelinase activity under normal control conditions, red line (bottom line): value of sphingomyelinase activity in the presence of FTY720.

FIG. 8 shows the effect of different sphingolipid metabolism inhibitor drug screenings (10 μM, 37° C., 24 h) on the intracellular C18 sphingomyelin (SM) content of MEF cells deficient in ATP13A2 in an initial screen for sphingomyelin regulatory response to potential drugs.

FIG. 9 shows the effect of different sphingolipid metabolic inhibitor drug candidates on the ability of the ATP13A2 KO disease model mice to move on a rotating rod in screen for neuromuscular impairment to define the motor regulatory response to potential drugs.

FIG. 10 shows differentially expressed genes associated with sphingolipid metabolism in the orbitofrontal cortex site of brain tissue of FTY720-treated ATP13A2 KO mice, obtained from triplicate treatments according to single cell gene analysis. Red (dark) color indicates high expression and blue (light) color indicates low expression.

FIG. 11 shows the results of lipidomic assays obtained according to the liquid-liquid chromatography and mass spectrometry method, with the ratio of disease ATP13A2 KO group/wild group in blue; the ratio of FTY720 treatment group/disease ATP13A2 KO group in red.

FIG. 12 shows the number of standing (vertical axis) of ATP13A2 KO disease model mice after oral administration of different doses of FTY720 (horizontal axis).

FIG. 13 shows the number of standing of ATP13A2 KO disease model mice before and after oral administration of FTY720 for different days (horizontal axis). 1-7 days with administration treatment on the disease model mice (shaded), 7-14 days with discontinuation treatment (unshaded), and 15-21 days with restored secondary administration treatment (shaded).

FIG. 14 shows the detection of movement function of C57BL/6 normal wild type (WT) mice treated with normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group) and other four groups, through rotating rod experiment after 5 days of intraperitoneal injection of FTY720 in rice (0.25 mg/kg/day, i.p.).

FIG. 15 shows the detection of movement function of C57BL/6 normal WT mice+normal saline control (C) group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group) and other four groups, through rotating rod experiment after 7 days of oral administration in mice (0.5 mg/kg/day, o.p.).

FIG. 16 shows the detection of movement function of C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group) and other four groups, through rotating rod experiment after 7 days of single administration (10 μg, i.c.v.) in the ventricles of mice.

FIG. 17 shows the detection of movement function of C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group) and other four groups, through standing experiment after 14 days of oral administration in the brain ventricles of aged mice.

FIG. 18 shows the detection of movement function of C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group) and other four groups, through rotating rod experiment after 14 days of oral administration in the brain ventricles of aged mice.

FIG. 19 shows a comparison of the standing condition of the same aged paraplegic mice with ATP13A2 KO before and after drug administration.

FIG. 20 shows a comparison of angle of the hind limb to ground (FBA) of the same aged paraplegic mice with ATP13A2 KO before and after drug administration.

FIG. 21 shows the diagrams of lipofuscin deposition (a-c) and statistical plots (d) in neurons of three groups of mice, including C57BL/6 normal WT mice+normal saline group (WT group), ATP13A2 KO neurodegenerative disease model mice+normal saline control group (KO+C group), and ATP13A2 KO neurodegenerative disease model mice+FTY720 group (KO+FTY720 group), after two weeks of drug administration, by taking brain tissues for transmission electron microscopy.

FIG. 22 shows the diagrams of axonal myelin sheath tearing (a-c) and statistical plots (d) of three groups of mice, including C57BL/6 normal WT mice+normal saline group (WT group), ATP13A2 KO neurodegenerative disease model mice+normal saline control group (KO+C group), and ATP13A2 KO neurodegenerative disease model mice+FTY720 group (KO+FTY720 group), after two weeks of drug administration, by taking brain tissues for transmission electron microscopy.

FIG. 23 shows the plots of neurotransmitter content in different brain tissue parts of mice from four groups, including normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group).

FIG. 24 shows stable conformations of sphingomyelinase (aSMA) bound by FTY720. (A): An equilibrium of FTY720 binding to sphingomyelinase is reached and stabilized within a 100 ns molecular dynamic simulation, indicating a direct action site of FTY720. FTY720 is shown in ball and stick with the carbon atoms colored yellow. Zinc ions are shown as purple spheres. The intermolecular hydrogen bond and electrostatic interactions are shown as blue and green dot lines, respectively. (B): Both bindings of the inhibitor FTY720 and the substrate sphingomyelin (SM) to the same structural pocket of aSMA (B), indicating a nature of competitive inhibition of sphingomyelinase by FTY720. Carbon atoms in FTY720 and SM are colored in yellow and grey, zinc ions are shown as purple spheres. (C) Molecular dynamic simulation (100 ns) shows no binding of fluoxetine (carbon atoms colored in cyan) to the substrate binding pocket of aSMA, indicating a negative control of binding analysis. Zinc ions are shown as purple spheres.

### DETAILED DESCRIPTION

The present disclosure is further described below in conjunction with the accompanying drawings and embodiments. The described embodiments allow the skilled person to more fully understand the present disclosure, but do not limit the present disclosure in any way.

Embodiment 1: neurodegenerative diseases with movement disorders having the following characteristic indicators: sphingomyelin accumulation in lysosomes and shortage in cell and tissue due to ATP13A2 mutation.

Experimental method: A cell model of neurodegenerative disease is constructed using human neuroblastoma cells (SH-SY5Y) cells transfected with ATP13A2 gene silencing shRNA-associated lentivirus for 48 hours. Lipids are extracted from SH-SY5Y cell lysosomes using a lipid extraction method, and appropriate internal standards are included. All lipid analysis is performed in electrospray ionization (ESI) mode using a liquid mass spectrometer.

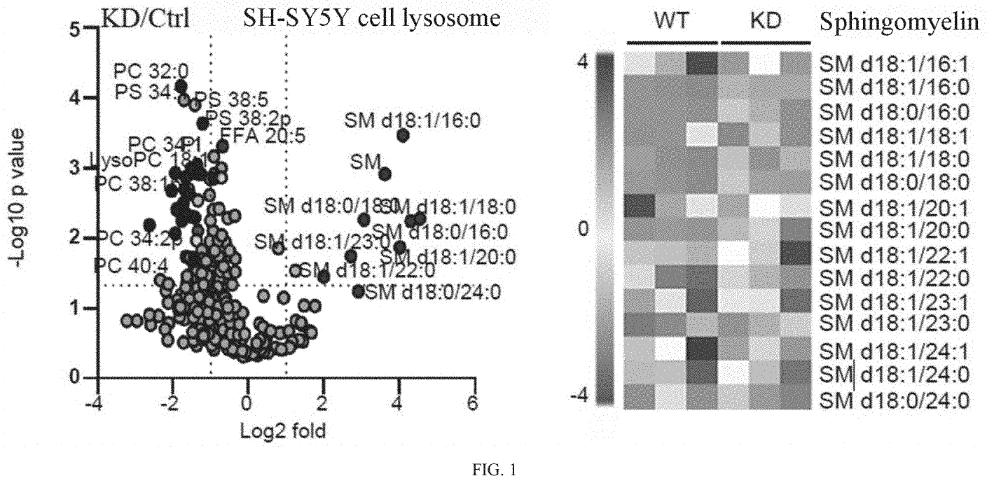

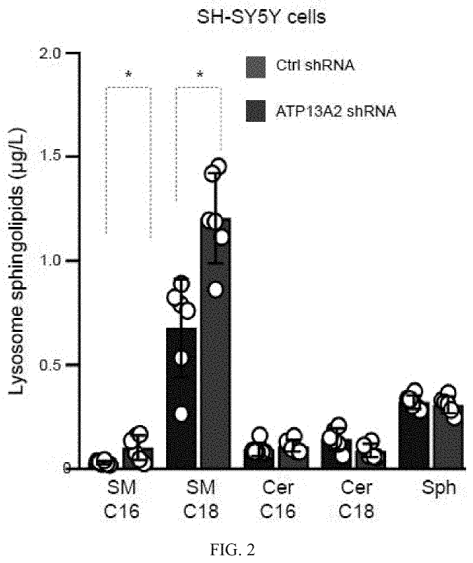

The results show that: As shown in FIG. 1, more than 200 lipids are analyzed and the results show (FIG. 1 left volcano plot) that sphingolipids are significantly increased specifically in this disease model. Further comparison of sphingolipids with different carbon chains and saturations shows (FIG. 1 right thermogram) that many sphingomyelin lipids are stored to varying degrees in the lysosomes of this cellular model of ATP13A2 deficient neurodegenerative disease. Thereafter, several major sphingolipids are measured in cultured cells with ATP13A2 KD. The results show (FIG. 2) that sphingomyelins (SM-C16 and SM-C18) are significantly increased in the lysosomes.

Embodiment 2: neurodegenerative diseases with movement disorders having specific lesion indicators 2: decreased sphingomyelins with or without increased ceramides. Experimental subject: brain tissue of ATP13A2 KO neurodegenerative disease model mice.

Experimental method: mouse brain tissue (substantia nigra, orbitofrontal cortex, hypothalamus) is taken and lipids are extracted from the brain tissue using a lipid extraction method and appropriate internal standards are added. All lipid analysis is performed in electrospray ionization (ESI) mode using a liquid mass spectrometer.

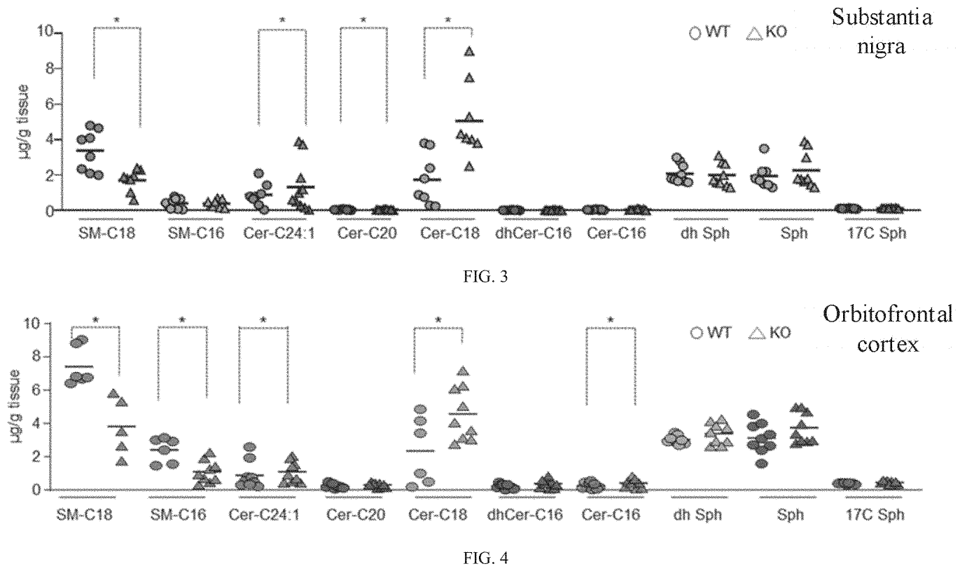

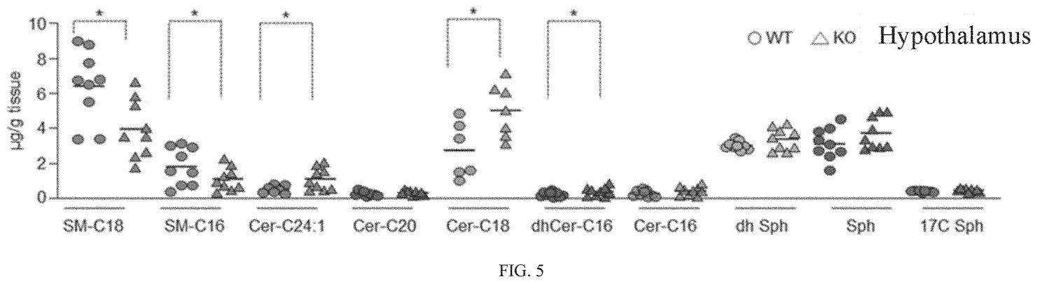

The results show that sphingolipid metabolism is abnormal in brain tissue of diseased mice, as shown in FIG. 3 (substantia nigra), FIG. 4 (orbitofrontal cortex), and FIG. 5 (hypothalamus), with a significant decrease in sphingomyelin (SM) in brain tissue (substantia nigra, orbitofrontal cortex, hypothalamus) of ATP13A2 KO mice compared to normal control mice, while ceramide content (Cer-C24:1, Cer-C20. Cer-C18) are significantly increased.

Embodiment 3: neurodegenerative diseases with movement disorders having specific lesion indicators 3: sphingolipid and ganglioside catabolism with or without increased cerebrosides.

Experimental method: Fibroblast MEF from wild-type and ATP13A2 KO neurodegenerative disease model mice fetal mice (15 days) are isolated, lipids are extracted from MEF cell lysosomes using a lipid extraction method, and appropriate internal standards are added. All lipid analysis is performed in electrospray ionization (ESI) mode using a liquid mass spectrometer.

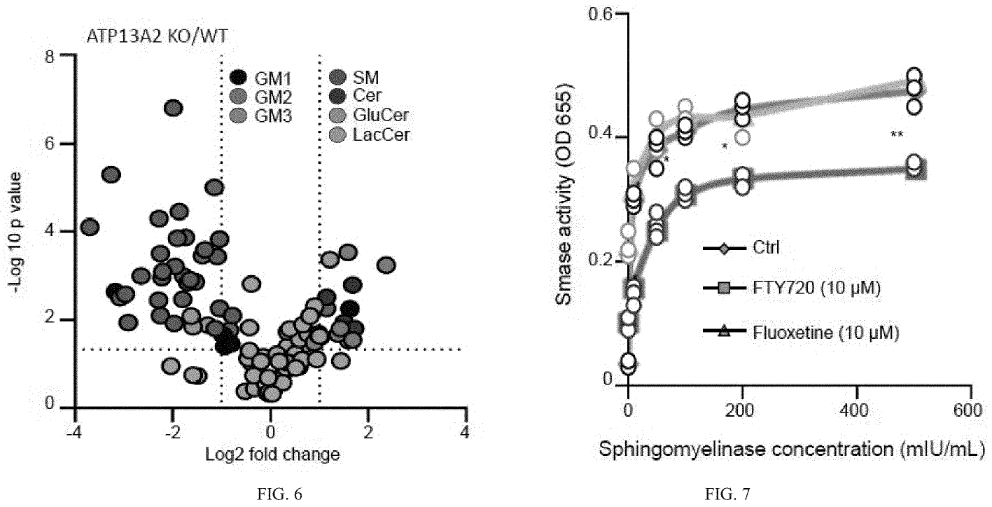

The results show (FIG. 6) that the content of sphingomyelin (SM) and gangliosides (GM1, GM2, GM3, indicated by blue tethered dots) etc. in the lysosomes of ATP13A2 KO MEF cells is significantly decreased whereas the content of ceramide (Cer) and cerebrosides (GluCer and LacCer) is significantly increased, suggesting that ATP13A2 in neuronal cells is involved in sphingomyelin and ganglioside catabolism and thus reduced in the pathological model.

Embodiment 4: Direct inhibition of sphingomyelinase by FTY720 Experimental method: FTY720 is purchased from Sigma, Cas No. 162359-56-0. Sphingomyelinase activity is determined using the Sphingomyelinase Assay Kit (ab138876) kit. The experiment is divided into two groups: normal sphingomyelinase activity assay group and sphingomyelinase activity assay group in the presence of FTY720. The results can be seen in FIG. 7: at different sphingomyelinase concentrations, there is a significant decrease in sphingomyelinase activity by FTY720 compared to the control group.

Embodiment 5: Drug candidate screening for sphingomyelin regulators in cellular mechanism of restoring sphingolipid homeostasis.

Experimental method: ten potential sphingolipid metabolic drugs reported in the literature for screening the modulatory effects on intracellular sphingolipids are selected, namely Fingolimod, Fluoxetine, Amitriptyline, Clomipramine, Desipramine, Siponimod, KRP-203, Amlodipine, Sertraline, Trimipramine, etc. MEF cells with disease-causing gene ATP13A2 KO are obtained using the disease model mouse embryos isolated and cultured. The MEF cells deficient in ATP13A2 are treated with the above ten drugs individually for 48 hours, and the intracellular content of sphingomyelin (SM C18) is measured by mass spectrometry.

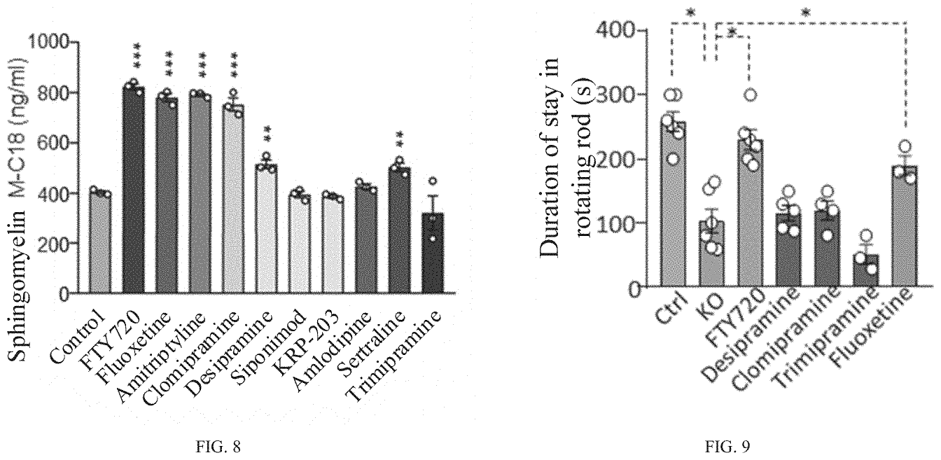

The results show that, as shown in FIG. 8, several putative sphingomyelin modulators have different degrees of upregulation effect on the intracellular sphingolipid content, among which, fingolimod, fluoxetine, amitriptyline, and clomipramine result in the most obvious elevation of intracellular sphingomyelin content.

Embodiment 6: Drug candidate screening of potential sphingomyelin regulatory drugs for reverting sphingomyelin related movement disorders in mice deficient in ATP13A2.

Experimental method: five drugs, including fingolimod, fluoxetine, clomipramine, desipramine, and trimipramine, are selected for oral administration (0.5 mg/kg) to diseased mice. The effect of the drugs on the movement function of the mice is examined one week after drug administration.

The results show that FTY720 improves the motor function of the ATP13A2 KO disease model mice among the five drugs with the most significant effect, as shown in FIG. 9.

Embodiment 7: Regulation of FTY720 on genes related to intracellular sphingolipid metabolism.

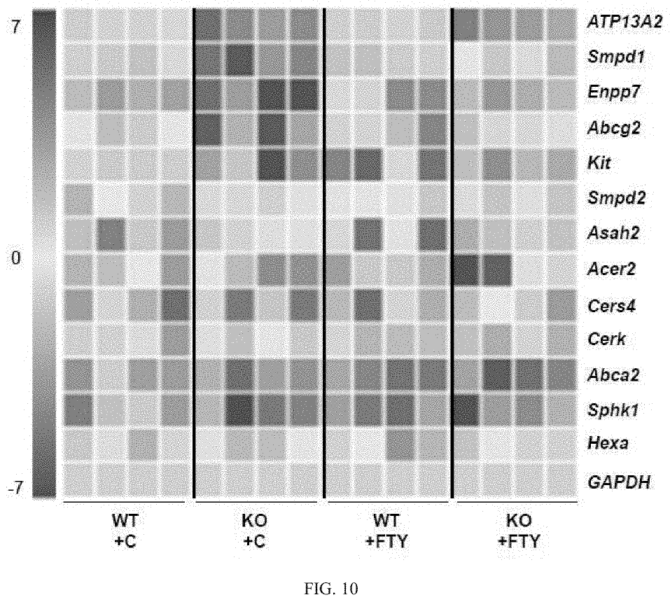

Experimental method: the experiment is divided into 4 groups: C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group). One week after intraperitoneal injection of FTY720 (0.5 mg/kg), samples are extracted from the orbitofrontal cortex area of mouse brain tissue, and Smpd1 Smpd2 Smpd3 Smpd4 Enpp7 Asah1 Asah2 Acer1 Acer2 Acer3 Cers1 Cers2 Cers3 Cers4 Cers5 Cers6 Col4a3 bp Cerk Gba Ugcg Degs1 Degs2 Sgpl1 Sptlc1 Sptic2 Sptic3 Abca1 Abca2 Abca7 Abca12 Abcc1 Abcg1 Abcg2 Sgms1 Sgms2 Cftr Sphk1 Sphk2 Slpr1 Kit Hexa Hexb Gal3st1 Gaphdh TERC TERT Ki67, etc. totally 47 genes related to sphingolipid metabolism are analyzed for comparison. The results show (FIG. 10) that after FTY720 treatment, genes such as smpd1 and enpp7 are upregulated and cers4, cerk, and sphk1 are downregulated in KO mice.

Embodiment 8: Regulation of FTY720 on intracellular sphingolipid content.

Experimental method: the experiment is divided into 4 groups: C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group). One week after intraperitoneal injection of FTY720 (0.5 mg/kg), mice are sampled from the striatum of brain tissue, lipids are extracted from the brain tissue samples using a lipid extraction method, appropriate internal standards are added. All lipid analysis is performed in electrospray ionization (ESI) mode using a liquid mass spectrometer.

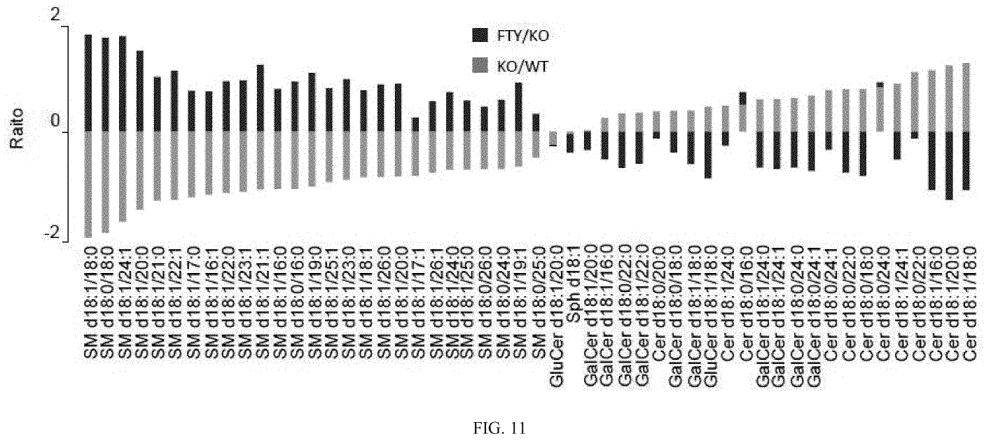

The results show (FIG. 11) that the sphingomyelin content decreases and the content of ceramide, glucose ceramide, and galactose ceramide increases in the striatal sites of the ATP13A2 KO neurodegenerative disease model mice. After FTY720 treatment, the content of sphingomyelin and ceramide in the disease model group mice mostly restores to the normal group level.

Embodiment 9: FTY720 increases the standing ability of paraplegia model mice in a dose-dependent manner

Experimental method: Spastic paraplegic ATP13A2 KO mice (KO mice) are divided into four groups and given normal saline or FTY720 dissolved in normal saline at 0.05, 0.1, and 0.5 mg/kg/day for oral administration, and the mice are tested in a standing test after 7 days of oral administration.

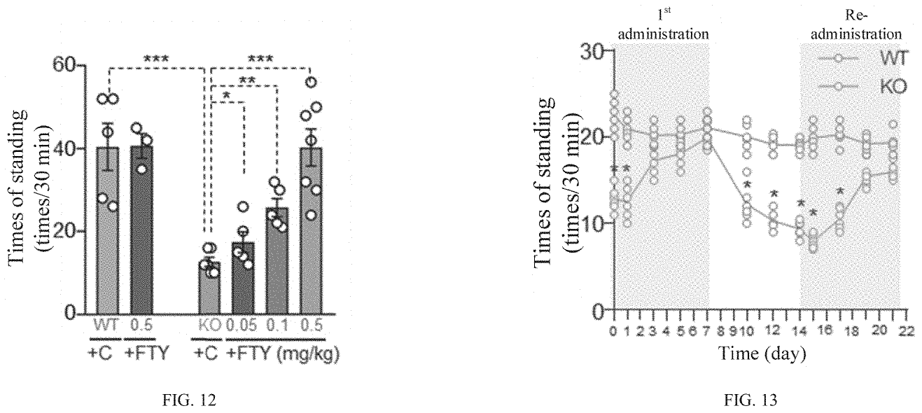

The results show that, as shown in FIG. 12, the number of standing per 30 minutes is significantly reduced in ATP13A2 KO paraplegic mice compared to control mice, while the number of standing of mice after different doses of FTY720 treatment is increased to different degrees in a dose-dependent manner of the ATP13A2 KO paraplegic mice.

Embodiment 10: FTY720 increases standing ability of paraplegia model mice in a time-dependent manner.

Experimental method: The experiment is divided into two groups: C57BL/6 wild normal mice (WT) and ATP13A2 KO spastic paraplegic model mice (KO), which are administered FTY720 0.5 mg/k/day orally at the same time, discontinued after 0-7 days of administration, and resumed after one week of discontinuation, during which the mice are tested for standing every other day.

The results show that the number of standing per 30 minutes in ATP13A2 KO paraplegic mice compared to control mice increases significantly with increasing administration time, while there is a gradual decrease in the number of standing of mice after drug discontinuation and a gradual increase in the number of standing by the resumption of administration on days 15-21 of the ATP13A2 KO paraplegic mice.

Embodiment 11: Intraperitoneal injection of FTY720 increases movement function in ATP13A2 KO disease model mice.

Experimental method: The experiment is divided into four groups: C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group). The mice are injected intraperitoneally with FTY720 (0.25 mg/kg/day, i.p.). 5 days later, the mice are tested for movement function using a rotating rod test.

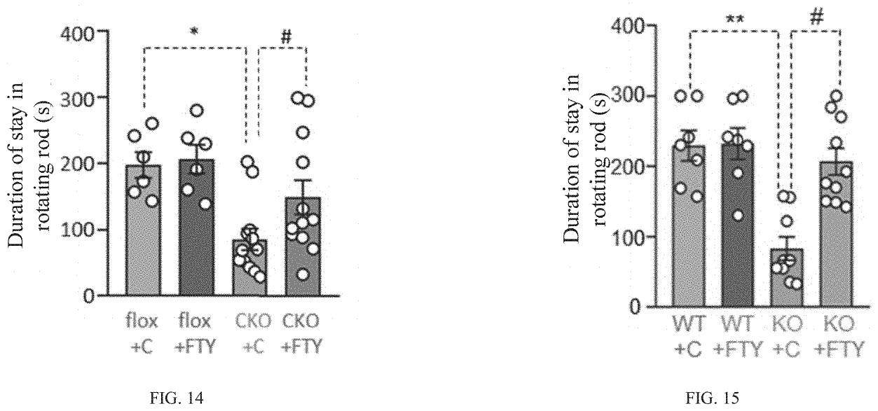

Experimental results: referring to FIG. 14, compared with the control group, the duration of stay in the rotating rod is significantly longer in the ATP13A2 KO neurodegenerative disease model mice after FTY720 treatment, indicating that the movement ability of the ATP13A2 KO neurodegenerative disease mice is significantly improved after the administration of the drug treatment.

Embodiment 12: Oral administration of FTY720 increases movement function in disease model mice.

Experimental method: The experiment is divided into four groups: C57BL/6 normal WT mice+normal saline control group (WT+C group), ATP13A2 KO neurodegenerative disease model mice+normal saline control group (KO+C group), normal WT mice+FTY720 group (WT+FTY720 group), ATP13A2 KO neurodegenerative disease model mice+FTY720 group (KO+FTY720 group). FTY720 dissolved in water is administered to mice orally administered (0.5 mg/kg/day), and one week later, the mice are tested for movement function using the rotating rod test.

Experimental results: referring to FIG. 15, compared with the control group, the duration of stay in the rotating rod is significantly longer in the ATP13A2 KO neurodegenerative disease model mice after FTY720 treatment, indicating that the movement ability of the ATP13A2 KO neurodegenerative disease mice is significantly improved after the drug administration treatment.

Embodiment 13: Ventricular injection of FTY720 increases movement function in disease model mice

Experimental method: The experiment is divided into four groups: C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group). A single administration (10 μg, i.c.v.) is administered to the mice by ventricular injection, and one week later, the mice are tested for movement function using the rotating rod test.

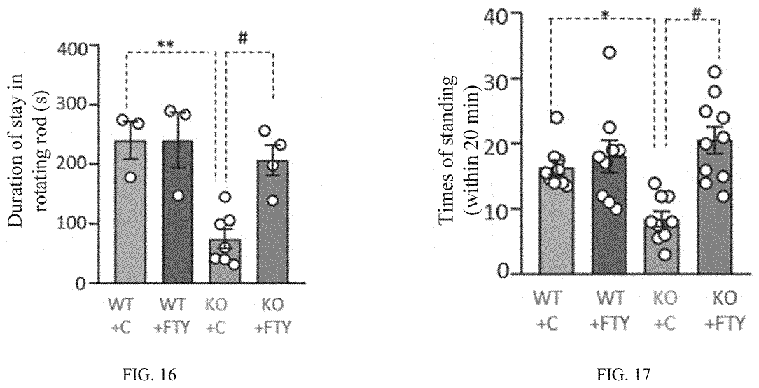

Experimental results: referring to FIG. 16, compared with the control group, the duration of stay in the rotating rod is significantly longer in the ATP13A2 KO model mice after FTY720 treatment, indicating that the movement ability of the ATP13A2 KO neurodegenerative disease mice is significantly improved after drug administration treatment.

Embodiment 14: Oral administration of FTY720 increases movement function in age-related disease model rice

Experimental method: The experiment is divided into four groups: C57BL/6 normal WT mice+normal saline control group (WT+C group), ATP13A2 KO aged disease model mice+normal saline control group (KO+C group), normal WT mice+FTY720 group (WT+FTY720 group), and ATP13A2 KO neurodegenerative disease model mice+FTY720 group (KO+FTY720 group). The mice are administered orally (0.5 mg/kg/day), and after two weeks, the movement function of the mice is examined using the standing test and the rotating rod test.

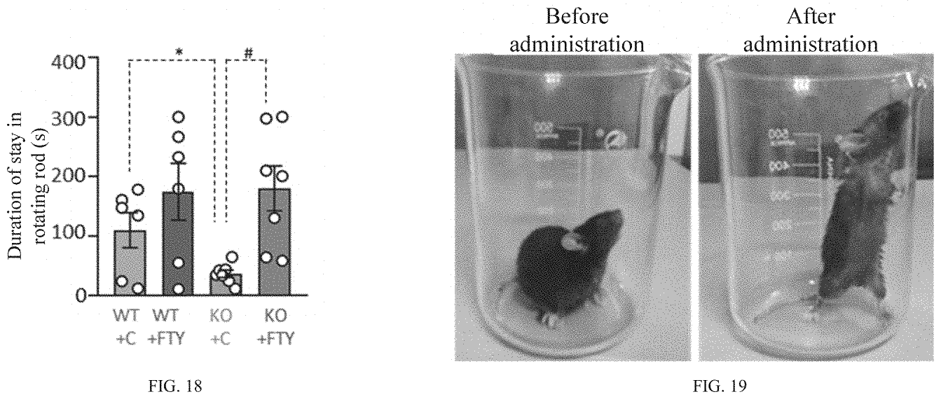

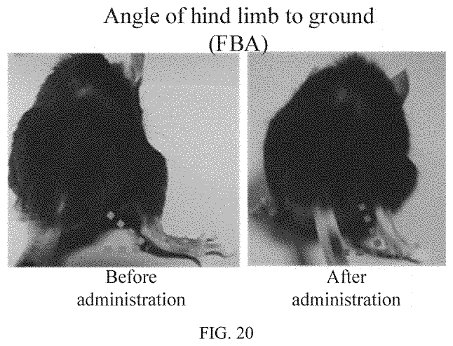

Experimental results: compared with the control group, there is a significant increase in the number of standing in the ATP13A2 KO model mice after FTY720 treatment (FIG. 17), while the duration of stay in the rotating rod is significantly longer (FIG. 18), indicating that the movement ability of ATP13A2 KO neurodegenerative disease mice is significantly improved after drug administration treatment. Meanwhile, the same aged ATP13A2 KO neurodegenerative disease mouse is placed in an upright glass beaker to observe its standing (FIG. 19). Before drug administration, the mouse almost always lays on the bottom of the beaker and rarely stands upright, and after drug administration, the aged ATP13A2 KO neurodegenerative disease mouse can be photographed to stand up by clinging to the beaker wall. The angle of the hind limb to the ground (FBA, foot-based angle) in the aged ATP13A2 KO paraplegic mouse is also measured. Based on the figures, it can be observed that a significant increase in the FBA angle in the aged mouse after drug administration, indicating that the drug can partially restore the phenotype of ATP13A2 KO neurodegenerative disease mice with trailing hind limbs in paraplegia (FIG. 20).

Embodiment 15: FTY720 attenuates the phenotype of lipofuscin deposition and axonal myelin sheath tearing in mouse brain tissue

Experimental method: The experiments are divided into three groups: C57BL/6 wild normal mice (WT), ATP13A2 KO neurodegenerative disease model mice+normal saline control (KO+C), and FTY720 treatment ATP13A2 KO mice (KO+FTY720). Mice are executed two weeks after oral administration, and brain tissue is removed and fixed for transmission electron microscopy analysis.

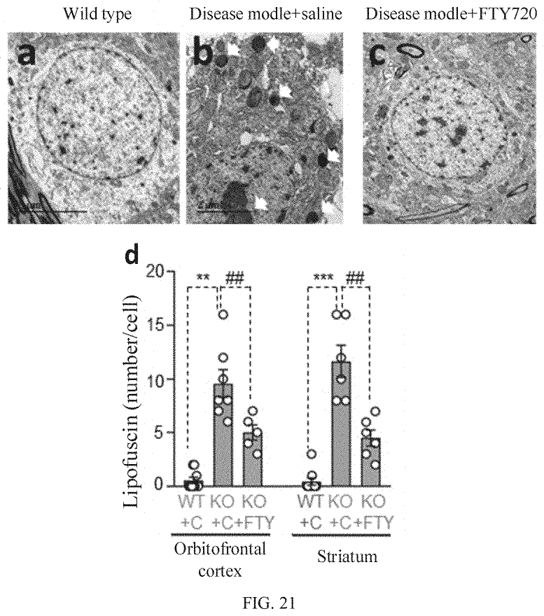

Result 1: referring to FIG. 21, compared to control mice (figure a), ATP13A2 KO neurodegenerative disease model mice have a significant amount of lipofuscin deposited in their brain tissue (figure b, indicated by red arrows), which is significantly reduced in neuronal cells after FTY720 treatment (figure c). Figure d shows the results of counting the number of intracellular lipofuscins for more than 100 cells, which shows that the number of lipofuscins is significantly reduced after FTY720 treatment of the ATP13A2 KO neurodegenerative disease mice.

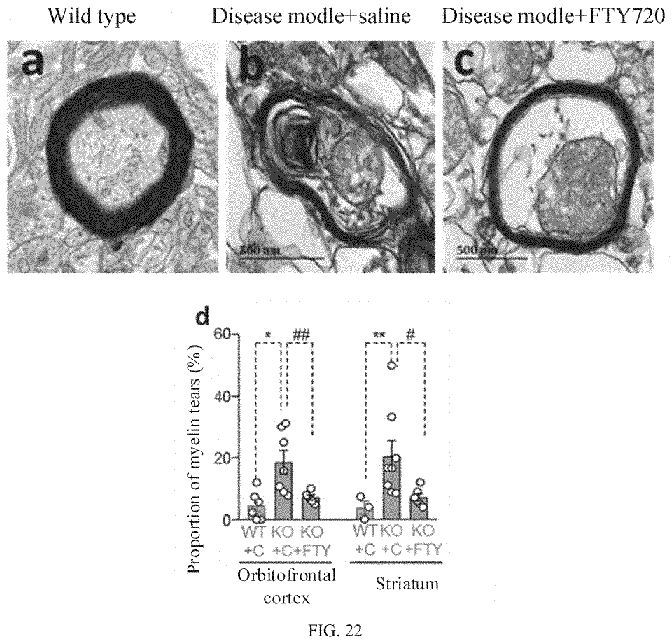

Result 2: referring to FIG. 22, compared to the control mice (figure a), the ATP13A2 KO neurodegenerative disease model mice also show significant tearing of axonal myelin sheaths in their brain tissue (figure b), the tearing of axonal myelin sheaths is significantly alleviated after FTY720 treatment, and the myelin sheath layer becomes denser (figure c). Figure d shows the results of counting the tears in more than 100 myelin sheaths, and it can be seen that the number of torn myelin sheaths is significantly reduced after FTY720 treatment of the ATP13A2 KO neurodegenerative disease mice.

Embodiment 16: FTY720 attenuates abnormal changes in brain tissue neurotransmitters in mice.

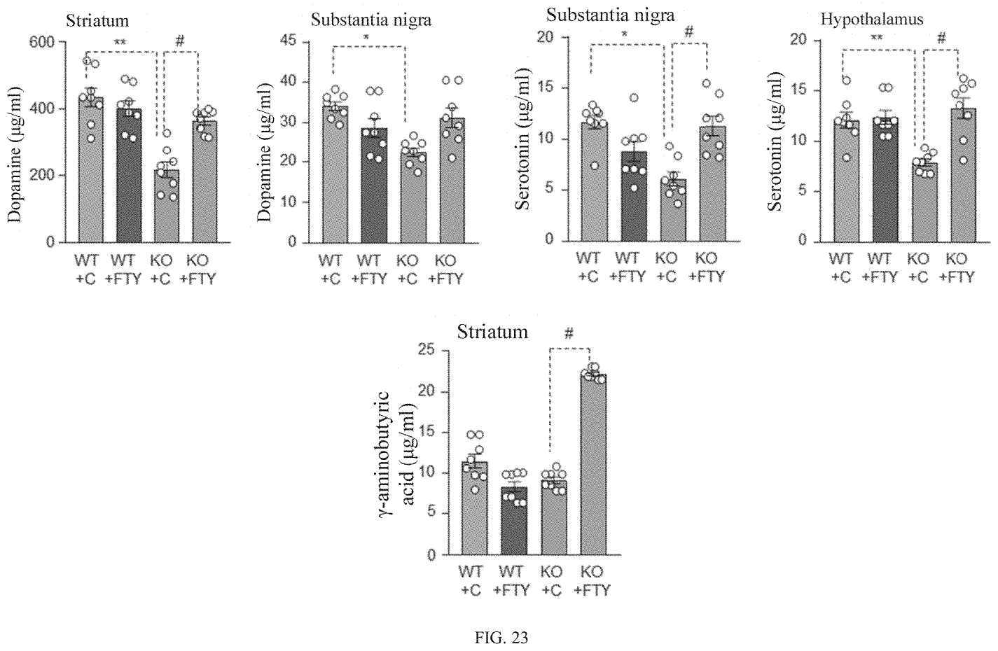

Experimental method: the experiment is divided into four groups: C57BL/6 normal WT mice+normal saline control group (WT+C group), ATP13A2 KO neurodegenerative disease model mice+normal saline control group (KO+C group), normal WT mice+FTY720 group (WT+FTY720 group), and ATP13A2 KO neurodegenerative disease model mice+FTY720 group (KO+FTY720 group). One week after mice are injected intraperitoneally with FTY720 (0.5 mg/kg), samples are taken from the striatum, hypothalamus, and substantia nigra of mouse brain tissue to extract neurotransmitters in the brain. Using a high performance liquid chromatography, neurotransmitters such as dopamine, serotonin, and γ-aminobutyric acid are analyzed. The results show (FIG. 23) that the levels of dopamine, serotonin, and γ-aminobutyric acid are decreased in the striatum and substantia nigra of the ATP13A2 KO neurodegenerative disease model mice. After FTY720 treatment, most of the neurotransmitter contents of mice in the disease model group are restored to the normal group level of the ATP13A2 KO neurodegenerative disease mice.

Embodiment 17: FTY720 binds to sphingomyelinase directly by molecular dynamic simulation.

Experimental method: Molecular dynamic (MD) simulations were performed with the Amber 20 package combined with the CHARMM36 force field. The binding free energies between FTY720 and the crystal structures of sphingomyelinase (aSMA, PDB ID: 2V3Y). For the comparison of binding affinities, the binding free energies were evaluated with MM/GBSA approach as descripted above, with the interior and exterior dielectric constants of 4.0 and 80 for aSMA-FTY720/SM/Fluoxetine.

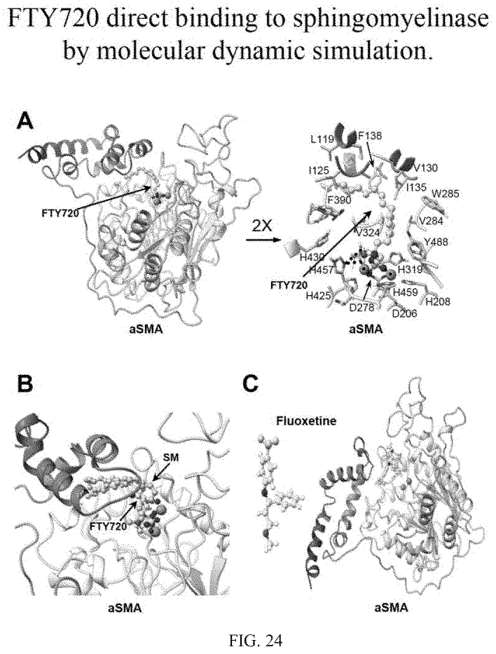

The results show (FIG. 24) a stable binding conformation of acidic sphingomyelinase (aSMA) by FTY720, indicating that FTY720 targets sphingomyelinase directly. FTY720 resembles the physical configuration of the sphingomyelinase bound by substrate sphingomyelin (SM), shares comparable binding free energies with sphingomyelin (SM) to bind the same site in aSMA, and differs from fluoxetine that does not form a stable complex with aSMA, indicating a nature of specifically competitive inhibition of sphingomyelinase by FTY720.

What is claimed is:

1. A medicinal use of fingolimod (FTY720) in prevention and treatment of neurodegenerative diseases caused by sphingolipid disorders; wherein the fingolimod with a chemical name of 2-amino-2-[2-(4-octylphenyl)]-1,3-propanediol hydrochloride is discovered as sphingomyelinase inhibitor, and the medicinal use is in a preparation of drugs against the neurodegenerative diseases caused by sphingolipid disorders.

2. The medicinal use according to claim 1, wherein the neurodegenerative diseases comprise early-onset Parkinson's syndrome and dementia (Kufor-Rakeb syndrome or KRS), hereditary spastic paraplegia (HSP, spastic paraplegia or SPG), amyotrophic lateral sclerosis (ALS, motor neuron disease or MND), corticobasal degeneration (CBD), spinal cerebellar ataxia (SCA), and neuronal ceroid lipofuscinosis (NCL) disease, as well as neuromotor diseases with disorders of sphingolipid metabolism due to reduced or deficient sphingomyelin or impaired recycling.

3. The medicinal use according to claim 1, wherein the drugs are made from an effective dose of FTY720 and pharmaceutically permissible excipients.

4. The medicinal use according to claim 3, wherein a total mass fraction of FTY720 in the drugs is 0.01-10%; drug powder of FTY720 is dissolved in 0.9% normal saline and administered in a concentration range of 0.01-5 mg/kg.

5. The medicinal use according to claim 1, wherein a route of administration of the drugs comprises oral administration, intraperitoneal administration, and ventricular injection administration.
